(19) United States
(12) Patent Application Publication
(10) Pub. No.: US 2015/0342686 A1
(43) Pub. Date: Dec. 3, 2015

(54) SYSTEMS AND METHOD FOR MICROACCESS TRANSESOPHAGEAL SURGERY

(71) Applicant: MICROACCESS INC., Weston, MA (US)

(72) Inventor: Sameh Mesallum, Boston, MA (US)

(21) Appl. No.: 14/721,492

(22) Filed: May 26, 2015

Related U.S. Application Data
(60) Provisional application No. 62/005,250, filed on May 30, 2014.

(51) Int. Cl. A61B 19/08; A61B 1/313; A61B 1/00; A61B 19/12; A61B 17/00

(52) U.S. Cl. CPC A61B 19/081; A61B 19/088; A61B 19/12; A61B 17/00234; A61B 1/00147; A61B 1/3132; A61B 1/00142; A61B 2019/082; A61B 2017/00278; A61B 2019/085

(57) ABSTRACT

Provided are minimally invasive approaches to perform translumenal surgery, including transesophageal surgery. In some embodiments, barrier devices and methodologies are provided to manage translumenal access to internal anatomy and surrounding structures to perform a body of surgical procedures (e.g., orthopedic, and neurosurgical procedures in the mediastinum, cervical, and thoracic cavities). In one embodiment, a translumenal barrier is used to demarcate a surgical access point within a patient's lumen (e.g., esophagus). In one example, the barrier is constructed of an elastic material (e.g., rubber) that includes a pre-defined opening biased to remain closed. The barrier covers a portion of the patient's lumen and manages access to the lumen at the pre-defined opening. In one example, surgical devices can be configured to deform the barrier to enter the pre-defined opening, and upon removal, the barrier returns to a closed position covering the body lumen and closing any opening the lumen.

### RELATED APPLICATIONS

This application priority under 35 U.S.C. §119(e) to U.S. Provisional Application Ser. No. 62/005,250 entitled “SYSTEMS AND METHOD FOR MICROACCESS TRANSESOPHAGEAL SURGERY,” filed May 30, 2014, which application is incorporated herein by reference in its entirety.

### BACKGROUND

Access to the human heart, the thoracic cavity, the neck structures, the cervical spine and the dorsal spine has always been difficult and a source of active research. More recently with the advancement in technology that has led to improved methods of minimally invasive surgery, interest in minimally invasive access to the heart and thoracic cavity have further increased. Some approaches that provide minimally invasive options for accessing the thoracic cavity (including e.g., transesophageal access) are described in, for example, U.S. Pat. No. 8,475,378 and U.S. Publication No. US 2010-0036197. Other groups are also exploring transesophageal surgery, as described for example in WO 2007/149588.

### SUMMARY

Stated broadly, various aspects relate generally to minimally invasive approaches to perform translumenal surgical, orthopedic, and neurosurgical procedures, for example, in the mediastinum, cervical, and thoracic cavities, for example, using a transesophageal approach. In some embodiments, surgical devices and methods are provided to manage transesophageal access to internal anatomy and surrounding structures to perform a body of surgical procedures. In some examples, the minimally invasive approaches enable surgical access and execution while the heart is beating with or without the need for general anesthesia.

According to one aspect, a translumenal barrier (also called a “barrier device” herein) is used to demarcate a surgical access point within a patient's lumen. In one example, the barrier is constructed of a barrier material (e.g., rubber) that covers a portion, for example, of the patient's esophagus where the barrier material includes a pre-defined opening. The barrier material can be biased to maintain the opening in a closed position. In one example, the barrier material around the pre-defined opening is elastic and configured to return to a closed position when not acted upon and maintain the closed position absent an application of force. In some examples, the barrier material can be constructed of an elastic material such that when deformed from an at rest configuration, the barrier material returns to the at rest configuration (i.e., closed opening). A first side portion and a second side portion of the barrier material can be configured to maintain the opening in the closed position based on respective elastic properties. Once the barrier is acted upon (e.g., by a surgical device) the elastic properties of the side portions can be overcome, opening the barrier.

For example, surgical devices can enter the thoracic cavity through the barrier using the pre-defined opening to perform a variety of surgical procedures. In some examples, a surgical device can be used to open the pre-defined opening and upon removal of the surgical device, the barrier seals any opening made in a body lumen.

In one example, the barrier comprises a rubber disk, oval, or sheet having a pre-defined slit through the barrier in a center portion of the barrier. According to one embodiment, opening of the pre-defined opening can occur through physical manipulation of the barrier (e.g., using a scope dilator) or via operation of opening mechanisms disposed within the barrier (e.g., actuators can be connected to each side of the opening to pull the barrier into an open configuration). In one example, the opening mechanism can include two actuators to apply opening pressure on opposite sides (e.g., opposite side portions) of the pre-defined opening. Once open, the barrier can provide surgical access, for example, to the patient's esophageal wall. According to some embodiments, the barrier is configured to return to a closed position sealing the esophagus and any surgical opening made in it responsive to cessation of any physical manipulation or responsive to the opening mechanisms being de-activate or reversed.

According to one embodiment, the barrier can be delivered to a selected site within a patient's esophagus and attached. In some embodiments, the barrier is placed at the selected site with the assistance of imaging techniques. In some examples, the barrier can be constructed of a translucent material to aid in imaging and/or visualization. According to one embodiment, the barrier can be removeably coupled to a distal end of a surgical device (e.g., a scope) having imaging components. The surgical device can be directed into a patient's mouth, for example, and into the patient's esophagus. Once in position, the barrier can be detached from the scope. In one example, the scope includes a bridge component configured to removeably couple to the barrier. In other embodiment, the barrier can include structures for removeably attaching to the scope or surgical device.

According to one embodiment, upon release of the barrier the scope can then be used to open the pre-defined opening on the barrier. For example, the scope can include a dilator which is used to open the pre-defined opening. In another example, the scope can be used to activate opening mechanisms disposed in the barrier. According to some embodiments, the scope can include a cutting instrument for opening a passage through the lumen wall (e.g., esophageal wall). Once the opening through the lumen wall is created, the scope and/or additional surgical devices can be used to perform surgical procedures, for example, in the thoracic cavity through the barrier and opening. Once the surgical procedure(s) are complete, the bias of the barrier to return to a closed position will cause it to seal over the opening in the esophageal wall. In some examples, the barrier can be attached to the esophageal wall such that the opening in the esophagus is likewise closed as the barrier operates to return to a closed position.

According to one aspect, a barrier device for performing minimally invasive translumenal surgery is provided. The barrier device comprises a first surface configured to attach to a portion of a body lumen, a second surface opposite the first surface and configured to be exposed to an interior of the body lumen, a pre-defined opening extending through the barrier device, a first side portion and a second side portion adjacent to opposite sides of the pre-defined opening, wherein the first and second side portions are biased to maintain the pre-defined opening in a closed position, wherein the pre-defined opening is configured to allow a surgical device to pass through the pre-defined opening from the second surface to the first surface and is configured to return to the closed position upon removal of the surgical device from the opening.

According to one embodiment, the barrier device is configured to removeably attach to a surgical device. According to one embodiment, the barrier device is configured to attach to an endoscope. According to one embodiment, the barrier device is configured to attach to a sterile sheath that covers the surgical device. According to one embodiment, at least a portion of the barrier device is configured to extend within the sheath of the surgical instrument. According to one embodiment, at least a portion of the barrier device is constructed of an elastic material configured to deform to an open position responsive to pressure and to return to the closed position upon removal of the pressure.

According to one embodiment, the barrier device comprises a translucent material. According to one embodiment, at least a portion of the barrier device is constructed of a translucent material. According to one embodiment, the barrier device comprises an adhesive layer configured to attach to the portion of the body lumen. According to one embodiment, the adhesive layer comprises an energy activated adhesive layer configured to attach to the portion of the body lumen in response to exposure to energy. According to one embodiment, the barrier device is configured to be attached to the portion of the body lumen such that when the pre-defined opening is in or returns to the closed position, the barrier device seals the body lumen from any incision in the portion of the body lumen. According to one embodiment, the barrier device further comprises an opening mechanism configured to mechanically overcome the bias of the first and second side portions so as to open the pre-defined opening.

According to one embodiment, the opening mechanism further comprises at least one motor connected to at least one of the first and second portion of the pre-defined opening. According to one embodiment, the opening mechanism further comprises a plurality of connectors, each connecting the at least one motor to a respective portion of the pre-defined opening. According to one embodiment, the opening mechanism is further configured to release the plurality of connectors such that the first and second side portions close the pre-defined opening. According to one embodiment, the second surface further comprises a plurality of sensor devices configured to trigger operation of the opening mechanism responsive to a detected electric field at the sensors. According to one embodiment, the barrier device further comprises activation sensors configured to detect deformation in the barrier device. According to one embodiment, the activation sensors are configured to detect physical contact between the barrier device and the surgical device.

According to one embodiment, the barrier is constructed and arranged of a uniform layer. According to one embodiment, the barrier is constructed and arranged of a uniform elastic layer. According to one embodiment, the barrier is constructed and arranged of a single uniform layer. According to one embodiment, the pre-defined opening is constructed and arranged within the uniform layer/singular uniform layer. According to one embodiment, the barrier is constructed and arranged of a first uniform surface, the first surface disposed away from the body lumen. According to one embodiment, the pre-defined opening is constructed and arranged to be accessible at the first uniform surface.

According to one aspect a kit for performing for performing minimally invasive translumenal surgery is provided. The kit comprises a barrier device, including a first surface configured to attach to a portion of a body lumen, a second surface opposite the first surface and configured to be exposed to an interior of the body lumen, a pre-defined opening extending through the barrier device, a first side portion and a second side portion adjacent to opposite sides of the pre-defined opening, wherein the first and second side portions are biased to maintain the pre-defined opening in a closed position, wherein the pre-defined opening is configured to allow a surgical device to pass through the pre-defined opening from the second surface to the first surface and is configured to return to the closed position upon removal of the surgical device from the opening; and a surgical device including attachment structures for connecting the barrier device and the surgical device.

According to one embodiment, the surgical device is an endoscope that includes a cutting instrument to create an incision in a portion of the lumen wall through the pre-defined opening. According to one embodiment, the attachment structures are configured to release the barrier device responsive to a control signal.

According to another aspect, a method of performing translumenal surgery is provided. The method comprises providing a barrier device having a pre-defined opening extending through the barrier device that is configured to remain in a closed position, inserting the barrier device into a body lumen, attaching the barrier device to a portion of a body lumen, inserting a surgical device into the body lumen and opening the pre-defined opening in the barrier device with the surgical device, creating an incision in the body lumen with the surgical device, performing a surgical procedure in an extra-lumen space, and removing the surgical device, returning the pre-defined opening in the barrier device to the closed position responsive to removal of the surgical device.

According to one embodiment, the method further comprises an act of deforming the pre-defined opening of the barrier device from the closed position with the surgical device. According to one embodiment, the method further comprises an act of providing an opening mechanism within the barrier device and activating the opening mechanism to transition the barrier from the closed position into an open position. According to one embodiment, the method further comprises an act of activating the opening mechanism responsive to detecting a position of the surgical device at the barrier device.

Various descriptions herein describe the use of the barrier within the lumen of the esophagus, in other embodiments the barrier can be introduced into other body lumens, and surgical procedures can be performed translumenally. In various embodiments, any body lumen or cavity can be accessed and operated on using the same principals and techniques. The barrier device allows for elongated surgical devices to be deployed across the lumen wall, to target extralumenal organs or structures for the performance of diagnostic and therapeutic procedures.

Still other aspects, embodiments and advantages of these exemplary aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. Any embodiment disclosed herein may be combined with any other embodiment. References to “an embodiment,” “an example,” “some embodiments,” “some examples,” “an alternate embodiment,” “various embodiments,” “one embodiment,” “at least one embodiment,” “this and other embodiments” or the like are not necessarily mutually exclusive and are intended to indicate that a particular feature, structure, or characteristic described in connection with the embodiment may be included in at least one embodiment. The appearances of such terms herein are not necessarily all referring to the same embodiment.

### BRIEF DESCRIPTION OF THE DRAWINGS

Various aspects of at least one embodiment are discussed below with reference to the accompanying figures, which are not intended to be drawn to scale. Where technical features in the figures, detailed description or any claim are followed by reference signs, the reference signs have been included for the sole purpose of increasing the intelligibility of the figures, detailed description, and claims. Accordingly, neither the reference signs nor their absence are intended to have any limiting effect on the scope of any claim elements. In the figures, each identical or nearly identical component that is illustrated in various figures is represented by a like numeral. For purposes of clarity, not every component may be labeled in every figure. The figures are provided for the purposes of illustration and explanation and are not intended as a definition of the limits of the invention. In the figures:

FIG. 1 illustrates an example of a surgical device and barrier for performing minimally evasive surgery, according to one embodiment;

FIG. 2 is a side cut out view of an example barrier, according to one embodiment;

FIG. 3 is cut out view of an example barrier, according to one embodiment;

FIG. 4 is view of a surgical device extended through a barrier, according to one embodiment;

FIG. 5 is a view of an example barrier, according to one embodiment;

FIG. 6 is an example process flow for minimally invasive surgery through a body lumen, according to one embodiment;

FIGS. 7A-C illustrate example embodiments of barriers, according to some embodiments;

FIGS. 8A-B illustrate example barriers in open and closed positions, according to some embodiments;

FIGS. 9A-B illustrate example embodiments barriers, according to some embodiments;

FIGS. 10A-B illustrate examples of a barrier, according to some embodiments;

FIG. 11 illustrates an example of a surgical device and connected barrier, according to one embodiment;

FIG. 12 illustrates an example surgical device with cutting implement, according to one embodiment;

FIG. 13 illustrates an example surgical device with a dilator, according to one embodiment; and

FIG. 14 is a schematic diagram of an exemplary computer system that may be configured to perform processes and functions disclosed herein.

### DETAILED DESCRIPTION

Stated broadly various aspects and embodiments are directed to minimally invasive translumenal surgical procedures. In various embodiments, a barrier is provided that manages sterile access through a body lumen. The barrier can be deployed on the end of a surgical device (e.g., a scope). In some embodiments, the barrier is releasably attached to the surgical device. When a desired location is reached, the barrier is attached to the body lumen (e.g., a patient's esophagus) and separated from the surgical device. Attachment can be made by suture, anchor, and/or adhesive, among other options. In one example, the barrier includes an adhesive layer that can be activated by the surgical device responsive to placement. For example, the surgical device can activate the adhesive layer chemically and/or by exposing the adhesive to light. In some embodiments, physical anchors and/or sutures can also be used in combination with the adhesive to secure the barrier to a body lumen. Once secured, pre-defined openings disposed in the barrier can be used to provide re-sealable access to the body lumen to generate a surgical opening and close the opening once a surgical procedure is complete.

In one example, the barrier has a pre-defined opening exposed to any lumen in which it is placed that is configured to remain closed unless acted upon. For example, a surgical device can be used to physically overcome the bias for the opening to remain closed to access the lumen to which the barrier is attached. In other examples, the barrier can include opening mechanisms that can be activated by the surgical device and/or via a control signal. Once open, the surgical device can cut through the body lumen to perform translumenal surgical procedures. In one embodiment, the barrier comprised a single contiguous layer having a pre-defined opening held closed by the bias characteristics of the material around the pre-defined opening. In another embodiment, the barrier is a unitary and uniform singular layer having a pre-defined opening.

At least some embodiments and methods include managing surgical procedures through the barrier and the lumen to which it is attached. In some embodiments, the barrier includes at least one pre-defined opening configured to remain closed unless acted upon. Further, the at least one pre-defined opening can be configured to return to the closed position and maintain the pre-defined opening in a closed position when at rest. Once released from an open position, the at least one pre-defined opening will close. Such configuration enables sterile access to a surgical field that is configured to automatically seal and protect the site of access.

Examples of the methods, devices, and systems discussed herein are not limited in application to the details of construction and the arrangement of components set forth in the following description or illustrated in the accompanying drawings. The methods and systems are capable of implementation in other embodiments and of being practiced or of being carried out in various ways. Examples of specific implementations are provided herein for illustrative purposes only and are not intended to be limiting. In particular, acts, components, elements and features discussed in connection with any one or more examples are not intended to be excluded from a similar role in any other examples.

Also, the phraseology and terminology used herein is for the purpose of description and should not be regarded as limiting. Any references to examples, embodiments, components, elements or acts of the systems and methods herein referred to in the singular may also embrace embodiments including a plurality, and any references in plural to any embodiment, component, element or act herein may also embrace embodiments including only a singularity. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms.

Various embodiments enable procedures that can safely and accurately create a translumenal (e.g., transesophageal) access into the mediastinum, the thoracic cavity and the cervical cavity. For example, the procedures can be carried out without the need for stopping the heart, or for cardiopulmonary bypass, general anesthesia, or gross or minor thoracotomy. Translumenal access as described herein can be used to perform a variety of diagnostic and therapeutic surgical, orthopedic and neurosurgical procedures.

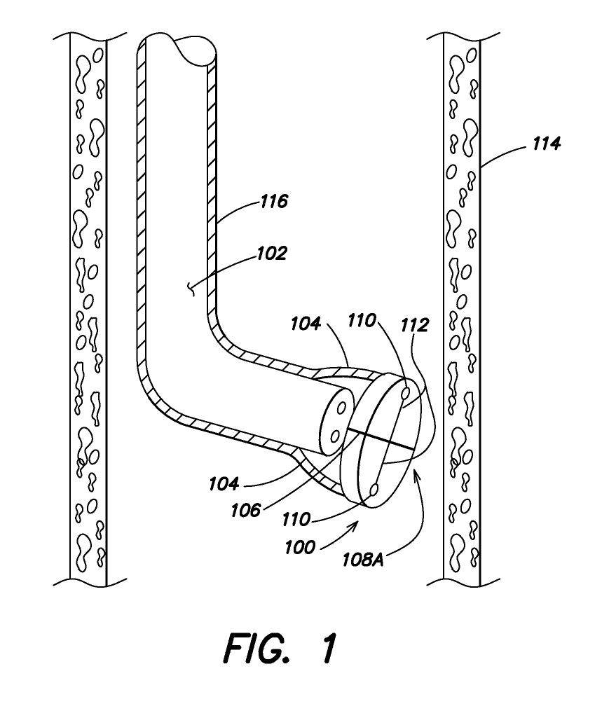

Shown in FIG. 1 is an example embodiment of surgical device 102 (e.g., scope) and barrier 100. The surgical device includes mating structures 104 for releasably attaching to the barrier 100. In one example, the barrier can be constructed of rubber or other elastomeric material. According to one embodiment, the barrier 100 includes a slit extending through the barrier 106. The barrier can be configured to maintain the slit in a closed position 108A until the slit is acted upon, either by the surgical device or through opening mechanisms 110. In some examples, opening mechanisms 110 can include paired actuators embedded in the barrier 100. Upon activation the actuators pull internal connectors 112 (e.g., strings) toward their respective actuator. The connectors are fixed at one end to respective sides of the slit 106, such that upon activation the actuators pull the slit 106 open. In another example screw drive motors may be used as opening mechanisms. Once activated, the motor pulls the edges of the slit 106 way from each other to provide an opening in the barrier 100.

According to one embodiment, the surgical device 102 can be used to position the barrier 100 in a patient's esophagus 114. To preserve the sterility of the surgical device, the device 102 can include a sterile sheath 116 that expands to maintain coverage over the surgical device as it progress into the patient's esophagus 114. The barrier 100 can be fixed to the esophagus so as to provide a sealed opening through which transesophageal surgical procedures may be performed.

According to some embodiments, the barrier device includes a pre-defined opening extending from a first surface of the barrier device to a second surface of the barrier device. The first surface can be constructed and arranged to be uniform when the pre-defined opening is in a close position. The second surface can be attached to a body lumen. The uniform first surface improves over other approaches, for example, as the barrier can remain in place in a body lumen while limiting any impediment to flow through the body lumen. In some embodiments, the barrier is constructed of a singular layer having a uniform first surface. The singular layer and uniform surface can facilitate long term placement of the barrier device in a body lumen, while minimizing any impact on normal bodily function in the lumen.

According to one aspect, the barrier device can be configured for facilitating the controlled and reversible creation of an opening in the wall of a body lumen, for example the esophagus, through which an endoscopic device can be inserted to perform surgery in adjacent tissues, particularly in the cervical and thoracic regions. The barrier device is a structure that covers part of the lumen walls of a hollow body organ or body cavity, in proximity to an organ or a structure outside the lumen. Various embodiments and examples are described in relation to the lumen of the esophagus. However, any body lumen or cavity can be targeted with similar techniques. In one example, the barrier device allows for elongated surgical devices to be deployed through/across the lumen wall, to target extralumenal organs or structures for the performance of diagnostic and therapeutic procedures.

According to one embodiment, the barrier can be held in place on a body lumen by an adhesive and/or mechanical attachment (e.g., sutures, anchors, etc.). The barrier typically remains in place before and after a surgical procedure to cover and protect the area of the body lumen that was opened to provide surgical access to an extralumenal organ and/or space.

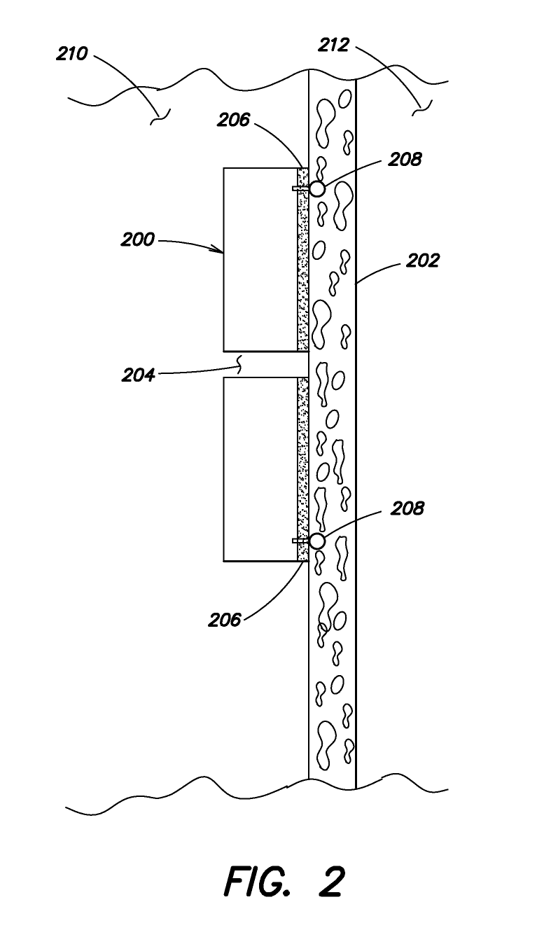

Shown in FIG. 2 is side cut out view of an example embodiment of a barrier 200 attached to a patient's esophageal wall 202. The barrier 200 includes a slit 204 or opening that extends the width of the barrier, which is configured to provide sealable access to the tissue of the esophageal wall 202. According to one embodiment, the barrier 200 is fixed to the esophageal wall 202 by an adhesive layer 206. In some embodiments, anchors 208 can also be used to fix the barrier 200 to the esophageal wall 202. Once in place the barrier 200 prevents bodily fluids or other contaminants from contacting the covered portion of the esophageal wall. In FIG. 2, slit 204 is illustrated as partially open, however, when the barrier is in its at rest configuration the slit 204 is closed. Once the barrier 200 is in place, a surgical device can be inserted into the slit 204 from the lumen of the esophagus 210. The surgical device can be used to cut an opening through the esophageal wall 202 to provide surgical access to the patient's extralumenal space 212, which can include organs and/or tissue.

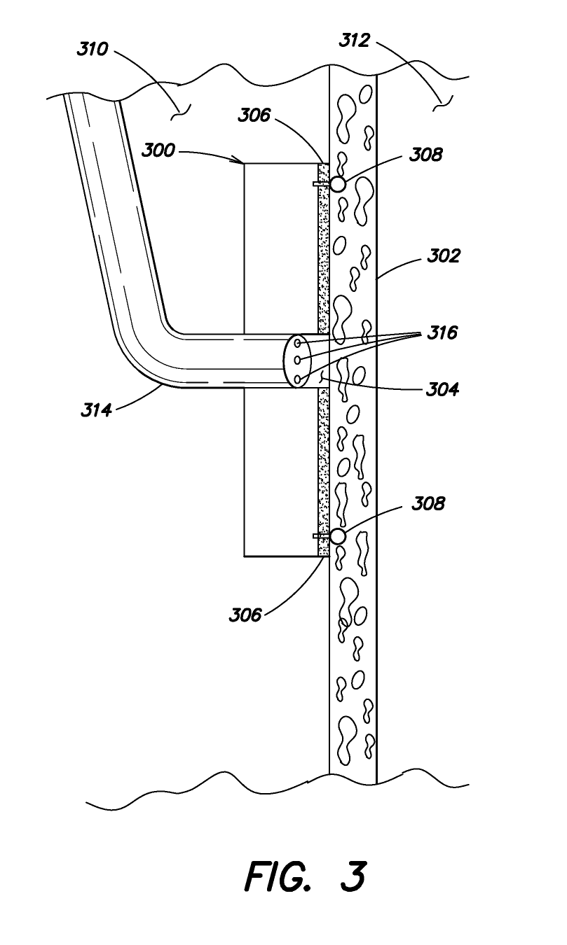

FIG. 3 shows a side cut out view of an example embodiment of a barrier 300 attached to a patient's esophageal wall 302. The barrier 300 includes a slit 304 or opening that extends the width of the barrier, which is configured to provide sealable access to the tissue of the esophageal wall 302. In FIG. 3, a surgical device 314 (e.g., a scope) is pushed into the opening distending the sides of opening until the barrier 300 is in an open position with the surgical device entering at least a portion of the pre-defined opening.

According to one embodiment, the barrier 300 is fixed to the esophageal wall 302 by an adhesive layer 306. In some embodiments, anchors 308 can also be used to fix the barrier 300 to the esophageal wall 302. Once in place the barrier 300 prevents bodily fluids or other contaminants from contacting the covered portion of the esophageal wall. According to one embodiment, a seal between the surgical device 314 and the barrier is maintained as the surgical device is advanced into the opening 304 from the lumen of the esophagus 310. The surgical device 314 can include a number of surgical instruments, and/or ports for delivery of chemicals, providing suction, irrigating a target area, etc. According to some embodiments, a cutting device can be extended from a port on the end of the surgical device (e.g., at 316) to create an opening through the esophageal wall 302 and into a space outside the esophagus 312.

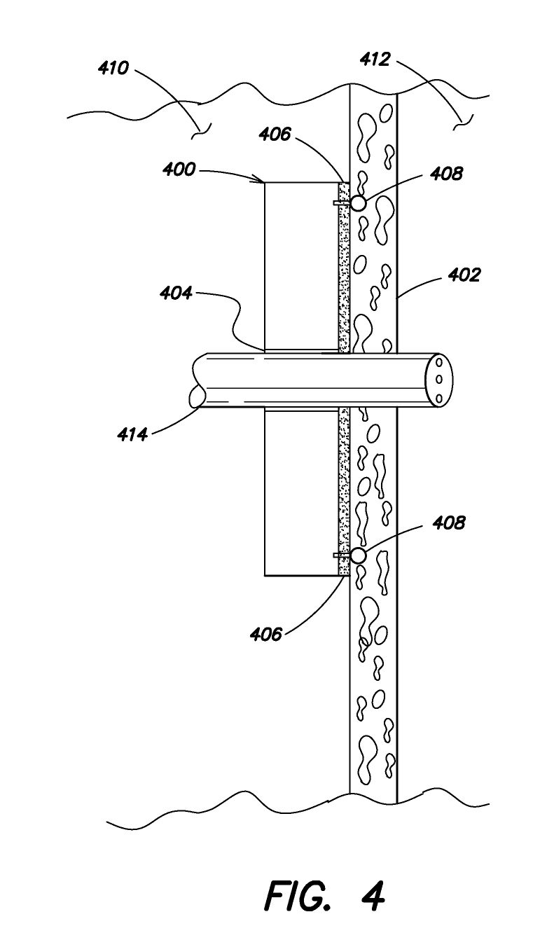

FIG. 4 shows a surgical device 414 extended through a barrier 400 in a cut out side view. The surgical device extends through a respective opening 404 of the barrier 400, and through the esophageal wall 402. Once through the esophageal wall 402 the surgical device can perform surgical operations within the patient's extralumenal space 412, which can include organs and/or tissue. In one example, the barrier 400 is fixed to the esophageal wall 402 by an adhesive layer 406. In some embodiments, anchors 408 can also be used to fix the barrier 400 to the esophageal wall 402. Once in place, the barrier 400 prevents bodily fluids or other contaminants from contacting the covered portion of the esophageal wall 402 and further prevents seepage through the opening cut into the esophagus. According to one embodiment, a seal between the surgical device 414 and the barrier is maintained as the surgical device is advanced into the opening 404 from the lumen of the esophagus 410 and into the extralumenal space 412.

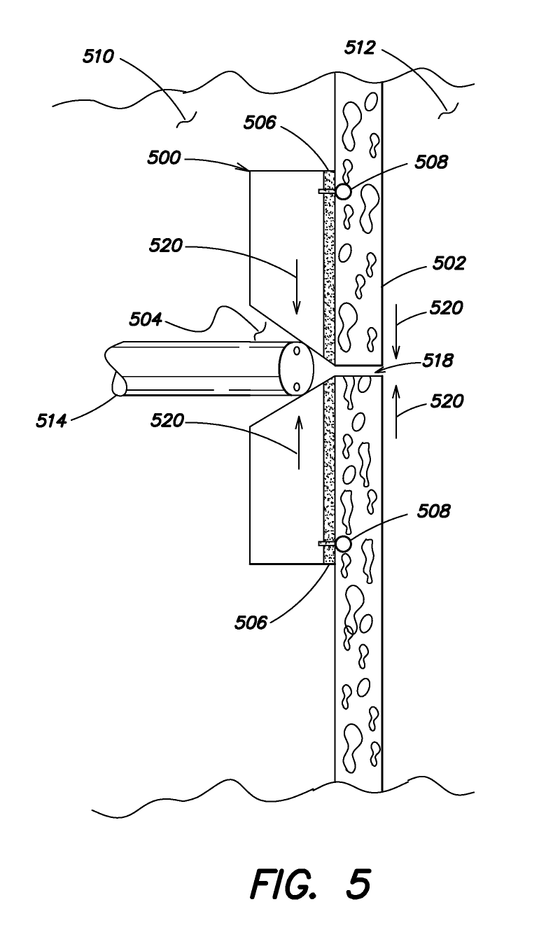

FIG. 5 shows a side cut out view of an embodiment of a barrier 500 automatically resealing the opening in the esophageal wall 502 and the opening 504 in the barrier as a surgical device 514 is withdrawn from the barrier 500. According to some embodiments, as the surgical device 514 is withdrawn the elastic properties of the barrier 500 cause the edges of the opening 504 to move together to close the opening 504 (shown by arrows 520). As edges of the opening 504 are drawn together the opening 518 in the esophageal wall 502 is likewise closed by virtue of the barriers attached to esophageal wall 502. An adhesive layer 506 and/or anchors 508 maintain a connection between the barrier 500 and the esophageal wall such that the opening 518 in the esophageal wall 502 closes with the opening 504 in the barrier 500. According to one embodiment, utilizing a self sealing barrier 500 enables transesophageal surgical access without contamination between the esophageal lumen 510 and the extralumenal space 512.

According to one aspect, minimally invasive procedures can be executed through any body lumen (e.g., the esophagus) using a barrier device. The barrier is fixed to the body lumen and used to provide a resealable opening to and through the body lumen. For example, an endoscopic instrument can be directed to a pre-defined opening in the barrier, pass through the opening, followed by passage through a portion of the body lumen to which it is attached (e.g., esophagus or other lumenal tissue). After removal of the instrument, the opening created in the body lumen can be closed automatically by the elastic properties of the barrier. The closing of the opening in the barrier likewise seals the opening made in the body lumen, and covers any incision.

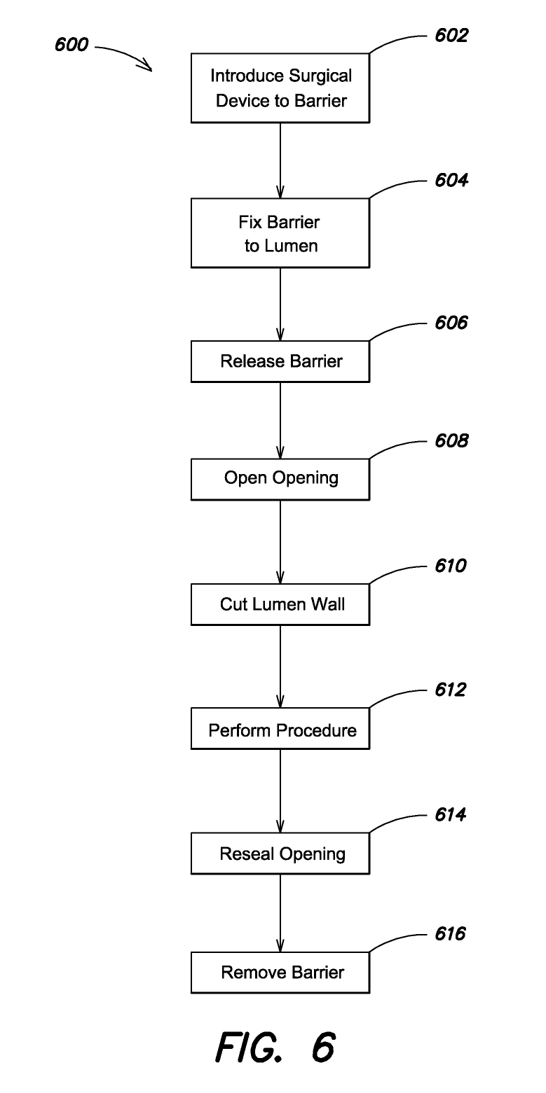

Various procedures use the barrier and a respective pre-defined opening. Shown in FIG. 6 is an example process flow 600 for performing minimally invasive surgery through a body lumen using a barrier. Process 600 begins at 602 with introduction of a surgical device into a natural body opening of a patient (e.g., mouth). According to some embodiments, the barrier is attached to the surgical device at a distal end. The surgical device can be used (e.g., at 604) to place the barrier on a desired position on the body lumen. Once correctly positioned, the barrier is fixed into position, for example, using an adhesive layer on the barrier. In some embodiments, the barrier included anchor structures to secure the barrier to the body lumen. For example, the barrier can be sutured to the lumen, welded to the lumen, stapled to the lumen, among other options.

At 606, the barrier is released from the surgical device and at 608 the barrier is manipulated to open a pre-defined opening in an interior portion of the barrier. In some examples, the surgical device can be advanced into the pre-defined opening. As the surgical device is advanced the barrier deforms to allow passage of the surgical device. In some examples, the barrier maintains a seal with the surgical device as it is advanced. Upon reaching the lumen wall, an incision can be made through the lumen wall (e.g., at 610) to provide surgical access to extralumenal space.

In other embodiments, the barrier can include opening mechanisms (e.g., motors) configured to open the pre-defined opening once activated. In one embodiment, the opening mechanisms can be activated at 608 to allow a surgical device to pass through the barrier. In some examples, motors are disposed in the barrier. The motors are connected to edges of the pre-defined opening such that the motors can open and/or close the opening. In one example, the barrier can include a switch on its surface to enable activation of the motors. A surgical device can be used to trigger the switch and open the barrier. In some embodiments, the switch can be responsive to removal of the surgical device, and close the opening as the surgical device is withdrawn from the barrier. In further examples, the surgical device can include an electrode specially configured to trigger the switch. At 608, the electrode can be extended from the device to trigger the switch opening the opening in the barrier.

As above, once a surgical device passes through the barrier and reaches the lumen wall, an incision can be made (e.g., at 610) and the surgical device can pass through the lumen wall and into the extralumenal space. In some embodiments, the elastic properties of the barrier can force the edges of the pre-defined opening to maintain a pressure seal against any device passing through the pre-defined opening. According to some embodiments, various surgical procedures can be performed at 612 (e.g., cardiac ablation, cardiac mapping, valve surgery, closure of septal defects, laser surgery, delivery of energy to the heart and related structure for pacing or to enhance contractility, delivery of drugs or genetic material, and delivery of surgical devices to the heart and related structures, among other options). Once the procedure is complete the surgical device is removed from the patient. As the surgical device is being removed, the elastic properties of the barrier cause the barrier to return to a closed position at 614. In some embodiments, the opening mechanisms can be used instead to close the opening as the surgical device is withdrawn. In further embodiments, the opening mechanism can assist the elastic properties of the barrier to close the opening as the surgical instrument is removed.

According to some embodiments, the pre-defined opening in the barrier is resealed at 614, as well any incision made in the body lumen. The incision made in the body lumen can be drawn closed by virtue of the adhesion between the barrier and the body lumen wall. Drawing the incision in the body lumen closed facilitates healing. Further, the barrier can be left in place to protect the incision site and further promote healing. In some embodiments, the barrier is sized and constructed to be non-occlusive so it may remain in place in the body lumen for the entire period of healing of the incision. After sufficient time has passed, the barrier may be removed from the patient. In one example, process 600 can include a removal step 616, where the adhesive layer is exposed to any one or more of light, heat, chemical, mechanical force, or laser energy to break the bond between the barrier and the body lumen wall. In one example, the application of energy to the adhesive layer breaks down the adhesive and releases the barrier from the lumen wall.

According to some embodiments, maintaining sterility at the incision site can be a factor in a positive outcome. To facilitate a sterile surgical environment, the surgical device and/or barrier can be contained in a sterile sheath or sleeve. The sleeve can be configured to surround the surgical device and/or barrier as they are advanced into a patient and delivered to a desired location. In some examples, a distal end of the sleeve is attached to a distal end of the surgical device and/or the barrier. A proximal end of the sleeve can include a pull string that allows an operator to extend the sleeve over the surgical instrument as it is advanced into a patient. In some embodiments, the sleeve is compressed into an according shape at a distal end of the surgical devices, and the pull string is configured to deploy the sleeve over the length of the surgical device as needed.

### Example Barrier Embodiments

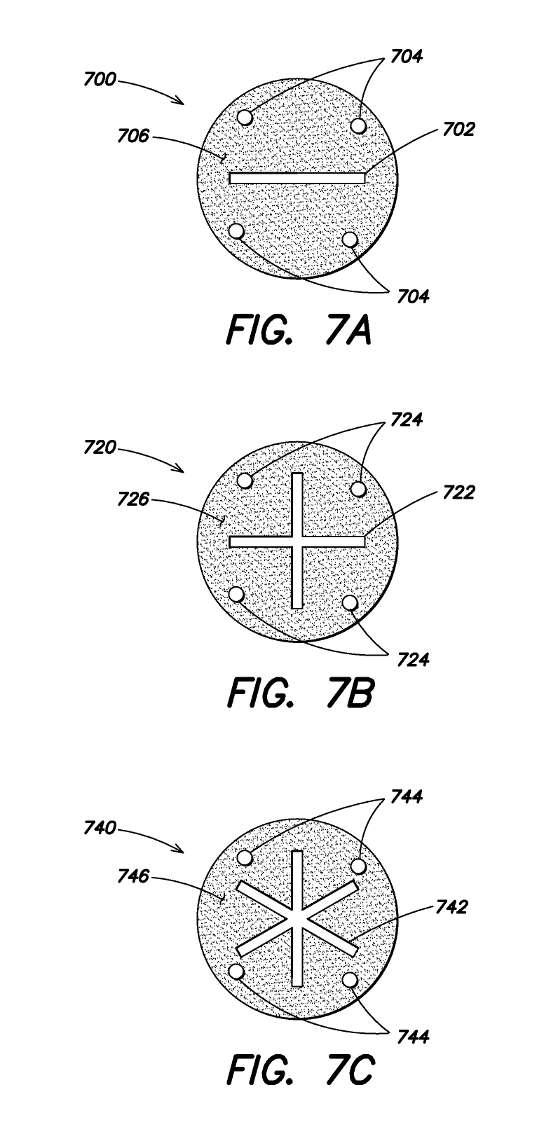

FIG. 7A shows an example embodiment of a barrier 700 having a pre-defined opening 702. In the figure, the opening dimensions are exaggerated for the purposed of illustration and clarity. In some embodiments, the barrier 700 can include anchors 704 built into the barrier. In further embodiments, the barrier can include anchor positions (e.g., at 704) to facilitate connecting the barrier to a body lumen via anchors. In some examples, the barrier includes an adhesive layer 706 for fixing the barrier to a body lumen. FIG. 7B illustrates another example embodiment of a barrier 720 having a pre-defined opening 722 configured in “x” pattern.” The barrier 720 can include anchors or anchor positions at 724. The barrier 720 can also include adhesive layer 726. The adhesive layer can be an energy or chemically activated glue, such that the barrier 720 adheres to the body lumen upon exposure to light, energy, and/or activating chemical. FIG. 7C illustrates another example embodiment of a barrier 740 having a pre-defined opening 742 in a star shape. In some embodiments, the barrier includes anchors or anchor positions at 744 and an adhesive layer 746.

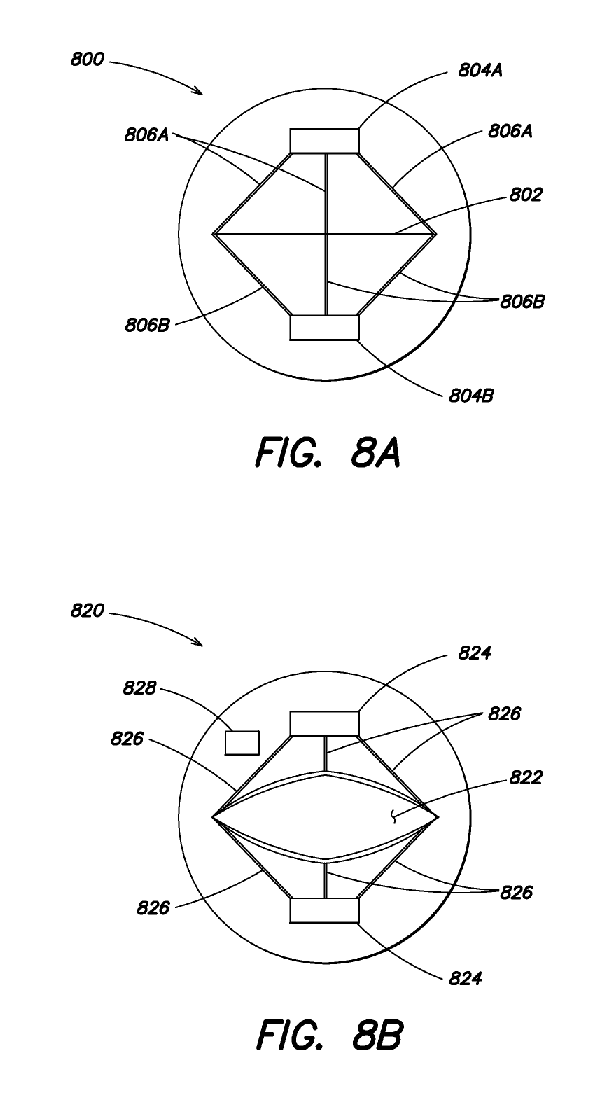

According to other embodiments, different configurations can be used for the pre-defined opening in the barrier. In some embodiments, the barrier can include additional structures to facilitate and/or manage the opening and closing of a respective pre-defined opening. Shown in FIG. 8A is an example embodiment of a barrier 800 having a pre-defined opening. The opening 802 is shown in a closed position. The barrier 800 includes actuators 804A and 804B which are configure to pull open the pre-defined opening 802 through connectors 806A and 806B respectively. The actuators 804A-B can be configured to operate in parallel, such that activation causes both actuators to pull on respective edges of the pre-defined opening 802. The actuators can be motors that operate on the connectors to open the barrier 800. In some examples, the motors can operate on strings or fibers connected to the edges of the pre-defined opening, to pull open the barrier. In other embodiments, the motors can be connected to drive shafts that operate to open and close the barrier. In further embodiments, the activations can operate on portions of the respective edges of the pre-defined opening and pull on the portions in sequence to partially open the predetermined opening as a surgical device is advanced into the opening.

FIG. 8B illustrates an example embodiment of a barrier 820 having a pre-defined opening 822 in an open position. The barrier 820 includes actuators 824 which have acted upon opening 822 to transition the barrier into an open position via connectors 826. In some embodiments, the barrier can include a sensor 828 to detect the presence of a surgical device at the opening 824. In example, the sensor is configured to detect deformation of the barrier 820 responsive to the surgical device being advanced into the opening 822. Once the deformation is detected by the sensor, the sensor triggers the actuators to assist in opening the barrier at 822. In other embodiments, the sensor can be configured to detect physical contact with a surgical device and trigger the actuators responsive to physical contact.

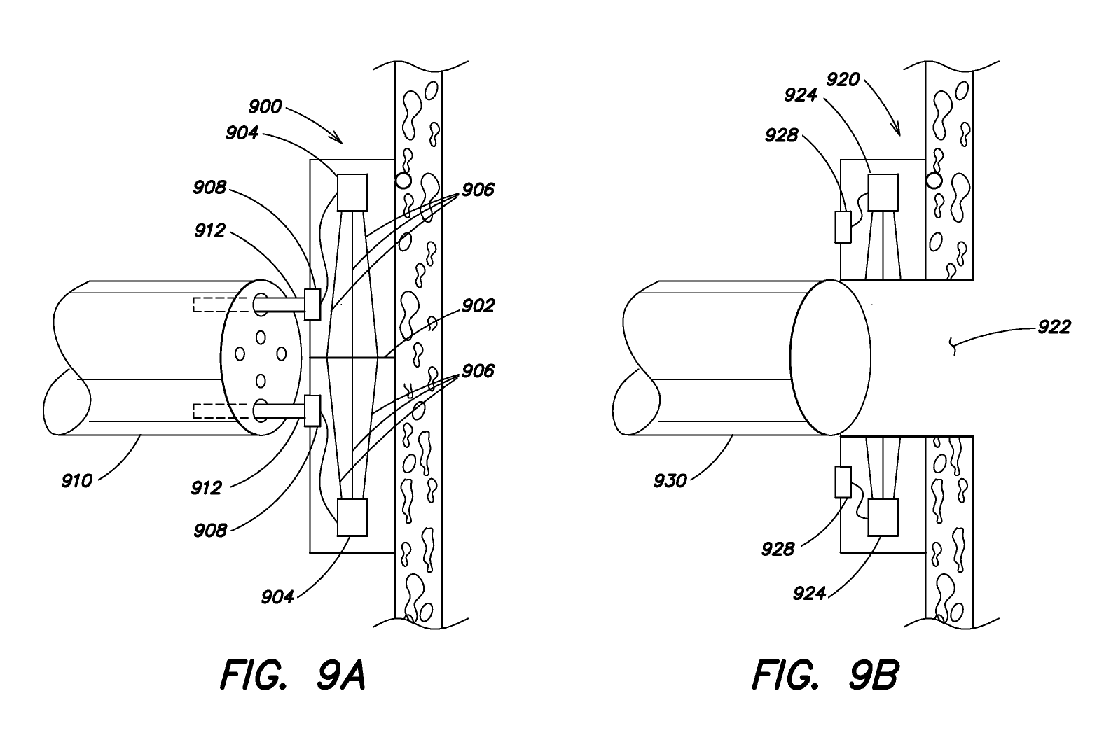

FIG. 9A illustrates an example embodiment of a barrier 900 having activations switches at 908 for triggering operation of embedded actuators 904. Once triggered the actuators can open a pre-defined opening 902 in the barrier 900. The actuators 904 can be connected to the opening 902 through connectors 906. In one embodiment, a surgical device 910 can include electrodes 912 specially configured to trigger switches 908. The electrodes 912 may be extended from ports in the surgical device 910 to trigger the switches.

In FIG. 9B, a surgical device 930 can be advanced through a barrier 920 and a pre-defined opening 922. The opening 922 is shown in an open position based on operation of actuators 924 triggered by respective switches 928.

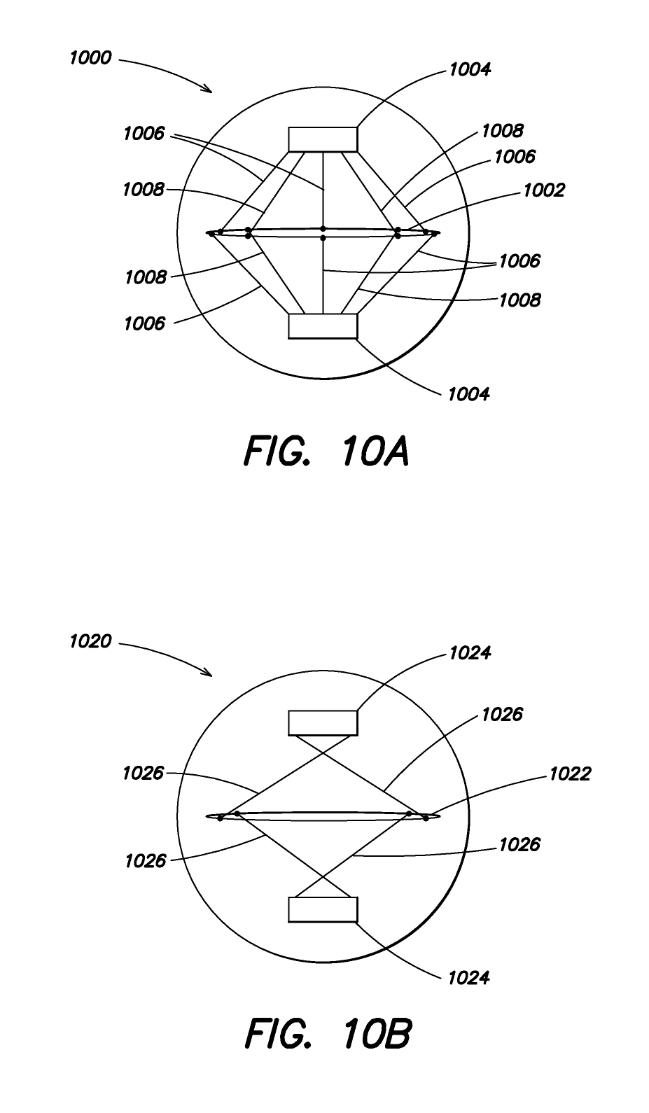

In FIG. 10A illustrated is another example embodiment of a barrier 1000. The barrier 1000 is constructed with opening mechanisms 1004 (e.g., actuators) that are configured to open and close a pre-defined opening 1002 of the barrier 1000. The opening mechanisms are configured to act on respective connectors at 1006 to open the barrier and respective connectors at 1008 to close the opening 1002 of the barrier. In FIG. 10B, an example embodiment of a barrier is illustrated with pre-defined opening 1002, actuators 1024. Only the closing connectors are shown at 1026. In some embodiments, the barrier 1020 can be constructed with only closing connectors, only opening connectors, and may include both opening and closing connectors.

### Example Surgical Fields

According to various embodiments, the barrier device can be inserted through a natural body opening of a patient (e.g., mouth, anus, vagina, ear, nose, etc.) to access body lumens. The barrier device can be placed within respective body lumens to manage sterile translumenal access for medical procedures. In some embodiments, the barrier device to can be used to manage translumenal access enabling procedures on the patient's heart and/or other organs or tissues in the intra-thoracic cavity using one or more devices introduced through the pre-defined opening in the barrier device and through the lumen to which it is attached.

The surgical procedures can include, for example, manipulation and/or surgical intervention of the patient's lungs, pleura, pulmonary vessels, bronchi, trachea and related structures. In other embodiments, the surgical procedures can include manipulation and/or surgical intervention on the patient's great vessels of the thorax, as well as, manipulation and/or surgical intervention on the patient's lymph nodes and lymphatic vessels of the thorax.

Further embodiments, can include surgical procedures for manipulation and/or surgical intervention on the patient's nerve roots and nerves of the thorax, manipulation and/or surgical intervention on the patient's ribs, intercostal tissues and structures, thoracic cartilaginous structures and muscles of the thoracic wall, manipulation and/or surgical intervention on the patient's thoracic and cervical vertebrae, intervertebral disks, foramina, nerve roots, paravertebral muscles, vertebral vessels and structures related to cervical and thoracic vertebrae.

In still other embodiments, the barrier and surgical devices can be configured for manipulation and/or surgical intervention on the patient's mediastinum and related mediastinal structures, or manipulation and/or surgical intervention on the patient's esophageal wall and paraesophageal structures. In other embodiments, other surgical procedures can be performed using the barrier device and associated surgical devices.

### Example Surgical Device Embodiments

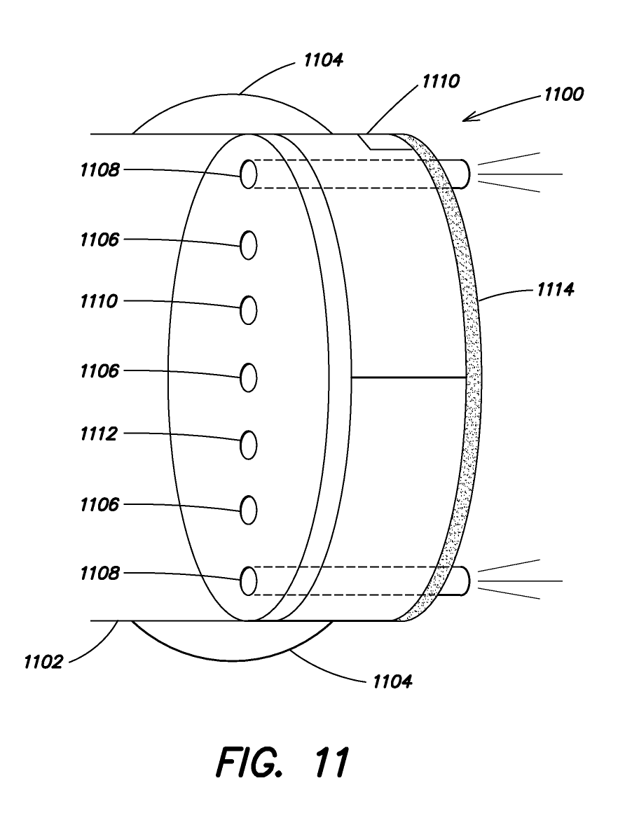

According to some embodiments, a barrier enables minimally invasive surgical procedures. The barrier device can be removeably attached to a distal end of a surgical device to facilitate placement, and the execution of the surgical procedure. FIG. 11 illustrates an embodiment of a surgical device 1102 and connected barrier 1100. The surgical device 1102 can be removeably connected to the barrier 1100 at holding bridge 1104. In some embodiments, the holding bridge 1104 can be configured to release the barrier, for example, responsive to a control signal or activation of a mechanical release. The surgical device 1102 can be specially configured based on the surgical procedure being performed. In some embodiments, the surgical device includes working channels, for example, at 1106. A variety of surgical devices can be extended from the working channels to perform surgical procedures (e.g., cutting devices, injection needles, ablation electrodes, among other options).

In some embodiments, the surgical device includes one or more washing channels (e.g., at 1108) for preparing a location for a surgical procedure. The one or more washing channels can be configured to deliver a sterile cleansing solution (e.g., an anti-septic solution). In some embodiments, the barrier 1100 can include openings to allow the washing channels from the surgical device to pass through the barrier. In further embodiments, the washing channels from the surgical device can be withdrawn once the barrier is in place, and the respective openings in the barrier can be biased to seal once the washing channels are removed. In other embodiments, the one or more washing channels can be positioned on the outer circumference of the barrier 1100. In further embodiments, the one or more washing channels are configured to sterilize the barrier on the lumen facing side.

In one embodiment, the surgical device can include imaging apparatus. At 1110, a lens is positioned at the distal end (distal being further away from the patient's natural body opening through which the device was introduced) of the surgical device 1102. The lens 1110 may be used to assist in placement of the barrier 1100 within a body lumen. In some embodiments, the surgical device may include a light source 1112. The light source 1112 can be used to facilitate imaging (e.g., by lens 1110). In further embodiments, the barrier can include an energy activated adhesive layer 1114. The light source 1112 can be configured to emit multiple wavelengths of light, and in particular to emit wavelengths of light to activate and/or break down the energy activated adhesive layer 1114.

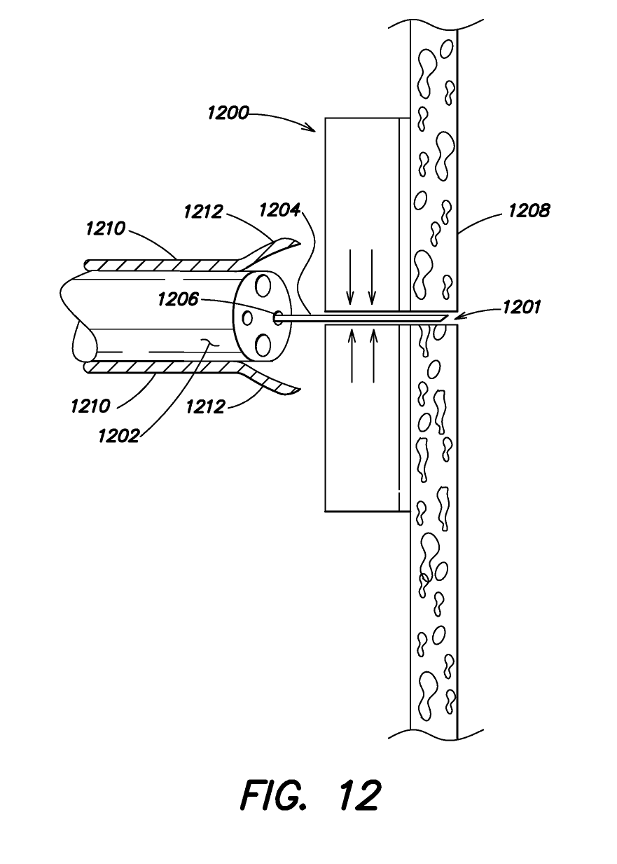

FIG. 12 illustrates an example surgical device 1202 with a cutting implement 1204 extended from a working channel 1206 in the surgical device 1202. The cutting implement 1204 is extended through the pre-defined opening 1201 in a positioned barrier 1200. According to some embodiments, a positioned barrier 1200 can be fixed to a lumen wall 1208. The barrier may be fixed in position using anchors between the barrier and the lumen wall and/or may use an adhesive layer to adhere the barrier to the lumen wall 1208.

According to some embodiments, the pre-defined opening 1201 is configured to return to a closed position. The bias to return to a closed position can be configured to maintain a seal between the cutting implement 1204 and the edges of the pre-defined opening 1201 as an incision is made in the patient's lumen wall 1208. In some embodiments, the surgical device 1202 is decoupled from the barrier 1200 by releasing a holding bridge 1212. The holding bridge can be released once the barrier is attached to the body lumen.

In some examples, the holding bridge 1212 can include structures that mate with apertures on the barrier 1200 (not shown). A variety of connectors can be used to releasably connect the surgical device to the barrier (e.g., key lock mechanisms, pressure fit connectors, magnetic attachments, etc.). In further embodiments, the surgical device can include a sterile sheath 1210 that is configured to cover the surgical device during a procedure. The sterile sheath can be bundled at the distal end (e.g., the end connected to the barrier). The sterile sheath bundle can be drawn over the surgical device as it is advanced into a patient's natural body opening. In some examples, the sterile sheath 1210 can extend to sides of the barrier 1200.

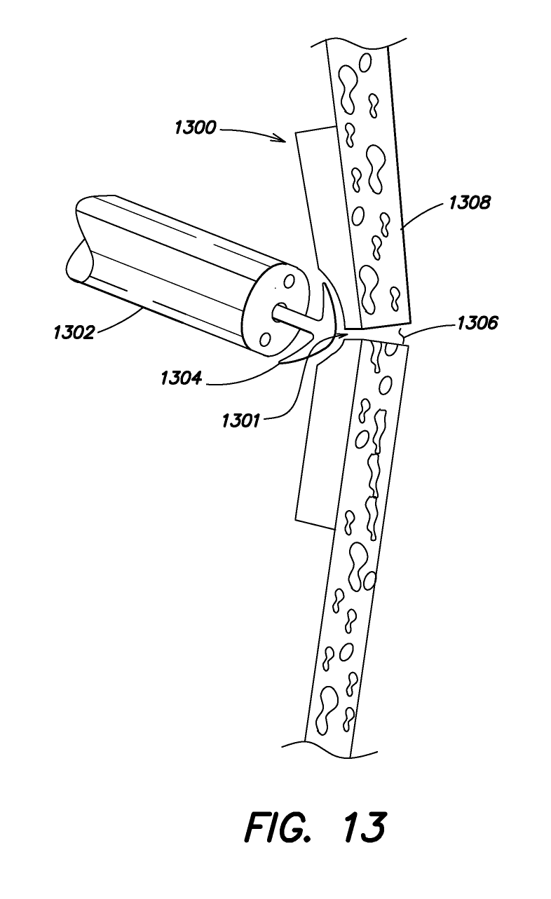

FIG. 13 illustrates another example surgical device 1302 with a dilator component 1304. The dilator component 1304 can be extended from the surgical device and used to expand the pre-defined opening 1301 in barrier 1300. As the opening 1301 is expanded, an incision 1306 made in the patient's esophageal wall 1308 can also be expanded. The expansion of the barrier 1300 and the incision 1306 enables the surgical device 1302 to pass through the pre-defined opening 1301 and the incision 1306 into the extra lumenal space.

As discussed above, various embodiments of the barrier are configured to maintain a pre-defined opening in a closed state unless acted open. A surgical instrument or opening mechanisms can be used to open the pre-define opening (e.g., using a dilator component 1304) so that the surgical instrument can pass through the barrier. Once the instrument is removed, the force keeping the pre-defined opening open is likewise removed and the barrier returns to its closed state, covering any opening in the body lumen. In one example, based on the barrier's attachment to the lumen wall, any opening cut into the lumen wall is closed as the edges of the pre-defined opening return to a closed state.

According to some embodiments, the barrier can also carry other devices, including for example a CCD chip or other chips that can convey images of the barrier's position in, for example, a patient's esophagus. The barrier can also be equipped with operative elements such as piezoelectric cells for ultrasound guidance either alone or combined with transesophageal ultrasound for stereoscopic recognition. The barrier can also be powered for operation of opening devices embedded with the barrier (e.g., motor based or other mechanical movement). The barrier may also include structures for heart treatment, cryotherapy, or magnetic or electromagnetic wave production/reception. Such enhancements are optional, and may instead be provide by other means in lumens that use barriers for the basic purpose of controlling instrument penetration and flow through the tissue of the wall of a lumen, particularly the esophageal lumen.

In another example, the barrier is positioned so that the pre-defined opening faces anteriorly to the heart, and is used with surgical devices for a variety of procedures including atrial mapping and ablation, treatment of arrhythmia, valvular heart disease treatments, occlusion of septal defects, etc. In other examples, the pre-defined opening may be facing anteriorly towards the lungs and anterior mediastinum for a variety of procedures both diagnostic or therapeutic, directly or in related structures, with the procedures including biopsy, tumor staging, imaging, injection, delivery of materials, cryotherapy, RF treatment, and laser treatment on tissues including lungs, great vessels, trachea, LN, esophagus, nerves, diaphragm, and lymphatics.

In further examples, the pre-defined opening may be positioned to access either or both of the cervical and thoracic regions of the esophagus, and can be position to face at least one of posteriorly, laterally, and anteriorly. The barrier may be used in procedures including but not limited to tumor excision or biopsy; placement of drugs, tissues, and radioactive materials; bronchial biopsy, airway bypass, manipulation of great vessels of the thorax, and pulmonary artery bypass. Additional procedures to be performed may include disk surgery, vertebral column surgery, spinal cord surgery, nerve root surgery, spinal and paraspinal muscle surgery, vascular surgery, oncologic surgery, laser surgery, delivery of energy to tissue, delivery of tissue or genetic material, delivery of surgical devices in general, delivery of cardiac pacemaker or diaphragmatic pace maker, and performance of procedures affecting the esophagus itself including fundoplication, and stomach pacemaker implantation. In further embodiments, the procedures to be performed can also include cardiac procedures including mapping, cardiac ablation, valve surgery, closure of septal defects, laser surgery, delivery of energy to the heart and related structure for pacing or to enhance contractility, delivery of drugs or genetic material, and delivery of surgical devices to the heart and related structures.

Other procedures can also include procedures on the lungs, bronchi, nerves, lymphatics, great vessels of the thorax, bony or cartilaginous structures, diaphragm, phrenic nerve, gastroesophageal junction and on the esophagus itself, including the delivery of an esophageal band for satiety or an intra-esophageal valve for reflux.

According to some embodiments, a surgical device coupled to the barrier can include imaging structures. The imaging structure can be used to facilitate placement of the barrier in a body lumen. In some examples, the barrier is constructed of a translucent material that enables imaging through the barrier. In further examples, an imaging apparatus can be extended into the barrier at a respective opening, such that the imaging apparatus extends through the opening to provide visual information on placement of a barrier (e.g., regardless of whether the barrier is translucent).

In further embodiments, the barrier can include processing capability and/or be connected to computer system that provides processing capability. In some examples, computer systems can be used to control opening and closing of pre-defined openings in barrier devices. For example, deformation sensors embedded in a barrier can deliver sensor information to a processor configured to analyze the sensor information to determine proximity of a surgical device. Responsive to determining that a surgical device is present at the pre-defined opening, the processor can be configured to open and/or close the pre-defined opening. In other embodiments, electrodes on the surgical device can be used to contact the barrier. Responsive to detecting contact at the barrier, the processor can be configured to trigger opening of the pre-defined opening. The absence of the electrodes can likewise be determined by sensor information received by the processor. Once the absence is determined the processor can be configured to trigger the pre-defined opening to close. In further embodiments, the actions triggered by the processor (e.g., open and/or close) can be responsive to positioning information of a surgical instrument within a pre-defined opening of a barrier. In some examples, sensors within the barrier can provide information on a penetration depth of a surgical device and control associated actuators accordingly.

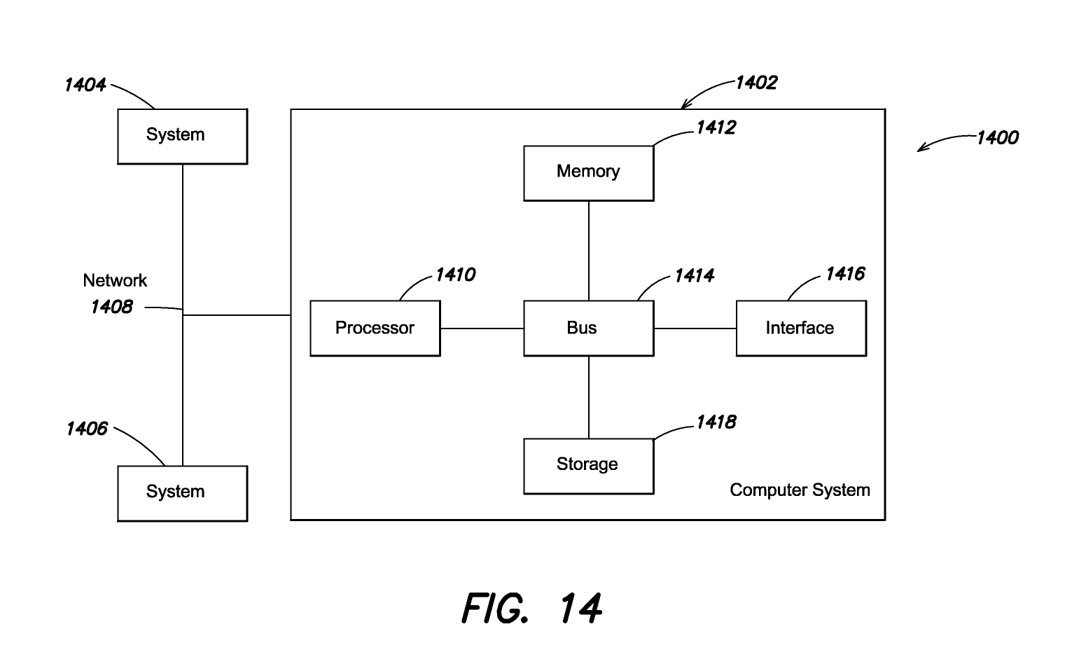

FIG. 14 is a block diagram of a special purpose computer system that can be specially programmed and/or configured to control actuators within a barrier device. The computer system can be configured to receive sensor information from sensors on or within the barrier devices and programmatically determine how to operate the actuators to open and/or close the barrier device in response.

Referring to FIG. 14, there is illustrated a block diagram of a distributed computer system 1400, in which various aspects and functions are practiced. As shown, the distributed computer system 1400 includes one or more computer systems that exchange information. More specifically, the distributed computer system 1400 includes computer systems 1402, 1404 and 1406. As shown, the computer systems 1402, 1404 and 1406 are interconnected by, and may exchange data through, a communication network 1408.

In some embodiments, the network 1408 may include any communication network through which computer systems may exchange data. To exchange data using the network 1408, the computer systems 1402, 1404 and 1406 and the network 1408 may use various methods, protocols and standards, including, among others, Fibre Channel, Token Ring, Ethernet, Wireless Ethernet, Bluetooth, IP, IPV6, TCP/IP, UDP, DTN, HTTP, FTP, SNMP, SMS, MMS, SS7, JSON, SOAP, CORBA, REST and Web Services. To ensure data transfer is secure, the computer systems 1402, 1404 and 1406 may transmit data via the network 1408 using a variety of security measures including, for example, TLS, SSL or VPN. While the distributed computer system 1400 illustrates three networked computer systems, the distributed computer system 1400 is not so limited and may include any number of computer systems and computing devices, networked using any medium and communication protocol.

As illustrated in FIG. 14, the computer system 1402 includes a processor 1410, a memory 1412, a bus 1414, an interface 1416 and data storage 1418. To implement at least some of the aspects, functions and processes disclosed herein, the processor 1410 performs a series of instructions that result in manipulated data. The processor 1410 may be any type of processor, multiprocessor or controller. Some exemplary processors include commercially available processors such as an Intel Xeon, Itanium, Core, Celeron, or Pentium processor, an AMD Opteron processor, a Sun UltraSPARC or IBM Power5+ processor and an IBM mainframe chip. The processor 1410 is connected to other system components, including one or more memory devices 1412, by the bus 1414.

The memory 1412 stores programs and data during operation of the computer system 1402. Thus, the memory 1412 may be a relatively high performance, volatile, random access memory such as a dynamic random access memory (DRAM) or static memory (SRAM). However, the memory 1412 may include any device for storing data, such as a disk drive or other non-volatile storage device. Various examples may organize the memory 1412 into particularized and, in some cases, unique structures to perform the functions disclosed herein. These data structures may be sized and organized to store values for particular data and types of data.

Components of the computer system 1402 are coupled by an interconnection element such as the bus 1414. The bus 1414 may include one or more physical busses, for example, busses between components that are integrated within a same machine, but may include any communication coupling between system elements including specialized or standard computing bus technologies such as IDE, SCSI, PCI and InfiniBand. The bus 1414 enables communications, such as data and instructions, to be exchanged between system components of the computer system 1402.

The computer system 1402 also includes one or more interface devices 1416 such as input devices, output devices and combination input/output devices. Interface devices may receive input or provide output. More particularly, output devices may render information for external presentation. Input devices may accept information from external sources. Examples of interface devices include keyboards, mouse devices, trackballs, microphones, touch screens, printing devices, display screens, speakers, network interface cards, etc. Interface devices allow the computer system 1402 to exchange information and to communicate with external entities, such as users and other systems.

The data storage 1418 includes a computer readable and writeable nonvolatile, or non-transitory, data storage medium in which instructions are stored that define a program or other object that is executed by the processor 1410. The data storage 1418 also may include information that is recorded, on or in, the medium, and that is processed by the processor 1410 during execution of the program. More specifically, the information may be stored in one or more data structures specifically configured to conserve storage space or increase data exchange performance.

The instructions stored in the data storage may be persistently stored as encoded signals, and the instructions may cause the processor 1410 to perform any of the functions described herein. The medium may be, for example, optical disk, magnetic disk or flash memory, among other options. In operation, the processor 1410 or some other controller causes data to be read from the nonvolatile recording medium into another memory, such as the memory 1412, that allows for faster access to the information by the processor 1410 than does the storage medium included in the data storage 1418. The memory may be located in the data storage 1418 or in the memory 1412, however, the processor 1410 manipulates the data within the memory, and then copies the data to the storage medium associated with the data storage 1418 after processing is completed. A variety of components may manage data movement between the storage medium and other memory elements and examples are not limited to particular data management components. Further, examples are not limited to a particular memory system or data storage system.

Although the computer system 1402 is shown by way of example as one type of computer system upon which various aspects and functions may be practiced, aspects and functions are not limited to being implemented on the computer system 1402 as shown in FIG. 14. Various aspects and functions may be practiced on one or more computers having a different architectures or components than that shown in FIG. 14. For instance, the computer system 1402 may include specially programmed, special-purpose hardware, such as an application-specific integrated circuit (ASIC) tailored to perform a particular operation disclosed herein. While another example may perform the same function using a grid of several general-purpose computing devices running MAC OS System X with Motorola PowerPC processors and several specialized computing devices running proprietary hardware and operating systems.

The computer system 1402 may be a computer system including an operating system that manages at least a portion of the hardware elements included in the computer system 1402. In some examples, a processor or controller, such as the processor 1410, executes an operating system. Examples of a particular operating system that may be executed include a Windows-based operating system, such as, Windows NT, Windows 2000 (Windows ME), Windows XP, Windows Vista or Windows 7 or 8 operating systems, available from the Microsoft Corporation, a MAC OS System X operating system available from Apple Computer, one of many Linux-based operating system distributions, for example, the Enterprise Linux operating system available from Red Hat Inc., a Solaris operating system available from Sun Microsystems, or a UNIX operating systems available from various sources. Many other operating systems may be used, and examples are not limited to any particular operating system.

The processor 1410 and operating system together define a computer platform for which application programs in high-level programming languages are written. These component applications may be executable, intermediate, bytecode or interpreted code which communicates over a communication network, for example, the Internet, using a communication protocol, for example, TCP/IP. Similarly, aspects may be implemented using an object-oriented programming language, such as .Net, SmallTalk, Java, C++, Ada, C# (C-Sharp), Objective C, or Javascript. Other object-oriented programming languages may also be used. Alternatively, functional, scripting, or logical programming languages may be used.

Additionally, various aspects and functions may be implemented in a non-programmed environment, for example, documents created in HTML, XML or other format that, when viewed in a window of a browser program, can render aspects of a graphical-user interface or perform other functions. For example, an administration component can render an interface in a browser to enable definition of contamination risks.

Further, various examples may be implemented as programmed or non-programmed elements, or any combination thereof. For example, a web page may be implemented using HTML while a data object called from within the web page may be written in C++. Thus, the examples are not limited to a specific programming language and any suitable programming language could be used. Accordingly, the functional components disclosed herein may include a wide variety of elements, e.g. specialized hardware, executable code, data structures or objects, which are configured to perform the functions described herein.

In some examples, the components disclosed herein may read parameters that affect the functions performed by the components. These parameters may be physically stored in any form of suitable memory including volatile memory (such as RAM) or nonvolatile memory (such as a magnetic hard drive). In addition, the parameters may be logically stored in a propriety data structure (such as a database or file defined by a user mode application) or in a commonly shared data structure (such as an application registry that is defined by an operating system). In addition, some examples provide for both system and user interfaces that allow external entities to modify the parameters and thereby configure the behavior of the components. In some embodiments, the system is configured to programmatically operate the opening mechanisms so that the pre-defined opening maintains physical connection to any surgical device being advanced into the opening. In other embodiments, the side not attached to the lumen may be opened faster than interior portions of the barrier so that a seal is maintain over the lumen until the surgical device is at least partially within the pre-defined opening.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A barrier device for performing minimally invasive translumenal surgery, the barrier device comprising:
a first surface configured to attach to a portion of a body lumen;
a second surface opposite the first surface and configured to be exposed to an interior of the body lumen;
a pre-defined opening extending through the barrier device,
a first side portion and a second side portion adjacent to opposite sides of the pre-defined opening, wherein the first and second side portions are biased to maintain the pre-defined opening in a closed position,
wherein the pre-defined opening is configured to allow a surgical device to pass through the pre-defined opening from the second surface to the first surface and is configured to return to the closed position upon removal of the surgical device from the opening.

2. The barrier device of claim 1, wherein the barrier device is configured to removeably attach to a surgical device.

3. The barrier device of claim 2, wherein the barrier device is configured to attach to an endoscope.

4. The barrier device of claim 2, wherein the barrier device is configured to attach to a sterile sheath that covers the surgical device.

5. The barrier device of claim 4, wherein at least a portion of the barrier device is configured to extend within the sheath of the surgical instrument.

6. The barrier device of claim 4, wherein the sterile sheath is coupled to an outer circumference of the barrier device and the sterile sheath is constructed and arranged to extend from a surgical field in a patient's esophagus to at least a natural body opening.

7. The barrier device of claim 1, wherein at least a portion of the barrier device is constructed of an elastic material configured to deform to an open position responsive to pressure and to return to the closed position upon removal of the pressure.

8. The barrier device of claim 1, wherein the barrier device comprises a translucent material.

9. The barrier device of claim 1, wherein the barrier device comprises an adhesive layer configured to attach to the portion of the body lumen.

10. The barrier device of claim 9, wherein the adhesive layer comprises an energy activated adhesive layer configured to attach to the portion of the body lumen in response to exposure to energy.

11. The barrier device of claim 9, wherein the barrier device is configured to be attached to the portion of the body lumen such that when the pre-defined opening is in or returns to the closed position, the barrier device seals the body lumen from any incision in the portion of the body lumen.

12. The barrier device of claim 1, wherein the barrier device further comprises an opening mechanism configured to mechanically overcome the bias of the first and second side portions so as to open the pre-defined opening.

13. The barrier device of claim 12, wherein the opening mechanism further comprises at least one motor connected to at least one of the first and second portion of the pre-defined opening.

14. The barrier device of claim 13, wherein the opening mechanism further comprises a plurality of connectors, each connecting the at least one motor to a respective portion of the pre-defined opening.

15. The barrier device of claim 12, wherein the opening mechanism is further configured to release the plurality of connectors such that the first and second side portions close the pre-defined opening.

16. The barrier device of claim 13, wherein the second surface further comprises a plurality of sensor devices configured to trigger operation of the opening mechanism responsive to a detected electric field at the sensors.

17. The barrier device of claim 16, further comprising activation sensors configured to detect deformation in the barrier device.

18. The barrier device of claim 16, wherein the activation sensors are configured to detect physical contact between the barrier device and the surgical device.

19. A kit for performing for performing minimally invasive translumenal surgery comprising:
a barrier device, comprising:
a first surface configured to attach to a portion of a body lumen;
a second surface opposite the first surface and configured to be exposed to an interior of the body lumen;
a pre-defined opening extending through the barrier device,
a first side portion and a second side portion adjacent to opposite sides of the pre-defined opening, wherein the first and second side portions are biased to maintain the pre-defined opening in a closed position,
wherein the pre-defined opening is configured to allow a surgical device to pass through the pre-defined opening from the second surface to the first surface and is configured to return to the closed position upon removal of the surgical device from the opening; and
a surgical device including attachment structures for connecting the barrier device and the surgical device.

20. A method of performing translumenal surgery, the method comprising:
providing a barrier device having a pre-defined opening extending through the barrier device that is configured to remain in a closed position;
inserting the barrier device into a body lumen;
attaching the barrier device to a portion of a body lumen;
inserting a surgical device into the body lumen and opening the pre-defined opening in the barrier device with the surgical device;
creating an incision in the body lumen with the surgical device;
performing a surgical procedure in an extra-lumen space; and
removing the surgical device;
returning the pre-defined opening in the barrier device to the closed position responsive to removal of the surgical device.
